(19) United States
(12) Patent Application Publication
(10) Pub. No.: US 2015/0258155 A1
(43) Pub. Date: Sep. 17, 2015

(54) PROCESS FOR OBTAINING CAFFEOYLQUINIC ACIDS-RICH EXTRACTS FROM HELIANTHUS ANNUUS

(71) Applicant: INDENA S.p.A., Milano (IT)

(72) Inventors: Ezio Bombardelli, Gropello Cairoli (IT); Fabrizio Corti, Milano (IT)

(21) Appl. No.: 14/433,351

(22) PCT Filed: Oct. 8, 2013
(86) PCT No.: PCT/EP2013/070928

§ 371 Date: Apr. 2, 2015

(30) Foreign Application Priority Data
Oct. 16, 2012 (IT) MI2012A001749

(51) Int. Cl. A61K 36/28; A23L 1/30

(52) U.S. Cl. CPC A61K 36/28; A23L 1/3002; A61K 2236/35; A23V 2002/00

(57) ABSTRACT

The present invention relates to extracts of deoiled Helianthus annuus seeds which are useful for the prevention and treatment of dyslipidaemia, hyperglycaemia and hypertension, metabolic syndrome and type 2 diabetes. The present invention also relates to the process for preparation of said extracts and compositions containing them. The extracts according to the invention, when added to carbohydrate-based foods, reduce the glycaemic index and postprandial absorption of glucose, and induce a modification of the lipid profile.

### FIELD OF INVENTION

The present invention relates to extracts of deoiled Helianthus annuus seeds which are useful for the prevention and treatment of dyslipidaemia, hyperglycaemia and hypertension, metabolic syndrome and type 2 diabetes. The present invention also relates to the process for preparation of said extracts and compositions containing them. The extracts according to the invention significantly reduce the postprandial and baseline blood glucose levels, and the blood triglyceride levels in overweight or obese patients. When the extracts according to the invention, complexed with macromolecules, are added to foods rich in starchy carbohydrates, their glycaemic index is reduced.

### PRIOR ART

Helianthus annuus extracts have been little used in traditional and allopathic medicine; however, Helianthus annuus seeds are widely used for the industrial production of oil, and the exhausted residue of the biomass is mainly used as forage in animal feed or biogas production.

Helianthus annuus oil is an excellent seed oil characterised by an appreciable content of glycerides, which modulate the intestinal absorption of fats. When the seeds are intact, or deprived of their outer shell, they contain variable amounts of caffeoylquinic acids in the form of mono- and diesters of quinic acid, of which chlorogenic acids form the preponderant part.

### DESCRIPTION OF THE INVENTION

It has now surprisingly been found that, thanks to the extraction process described below, it is possible to obtain extracts characterised by a high content of caffeoylquinic acids, which possess potent hypoglycaemic activity on the postprandial and baseline blood glucose levels.

The present invention therefore relates to Helianthus annuus extracts, the process for their preparation, and compositions containing them.

The process according to the invention comprises:

a) extraction of industrial residues of Helianthus annuus with aqueous mixtures of aliphatic alcohols;

b) concentration under vacuum of the water-alcohol solution from step a) until complete elimination of the alcohol solvent, and filtration of any insoluble matter and residual fatty phases;

c) adjustment of the pH of the aqueous solution from step b) to values around 4.5±1;

d) ultrafiltration of the aqueous solution from step c) through a 400 Da organic membrane;

e) chromatography or nanofiltration of the solution from step d);

f) concentration of the retentate from step e) under vacuum or by atomisation.

In step a), “industrial residues of Helianthus annuus” means extracts of Helianthus annuus seeds obtained by hot extraction with hexane followed by elimination of the solvent (“desolvation”) at temperatures exceeding 100° C.

According to a preferred aspect of the invention, the extraction of step a) is performed with aqueous mixtures of ethanol/water, preferably 80% v/v, in the presence of organic or inorganic acids able to maintain a pH of less than 2, preferably dilute sulphuric acid, until the mono- and dicaffeoylquinic acids are exhausted.

According to a preferred aspect of the invention, in step c), the pH of the aqueous solution is adjusted to values around 4.5±1 with calcium carbonate.

The aqueous solution originating from step c) undergoes absorption resin chromatography using a polystyrene resin and/or an ion exchange and absorption resin or nanofiltration on ceramic membranes with a 400 to 600 Da cut-off, to remove salts and undesirable low-molecular-weight products. The retentate retains caffeoylquinic acids, while salts and sugars remain in the permeate.

The process of the invention is of particular industrial interest, as the availability of biomasses is substantially unlimited and available at negligible cost, with evident benefits to the economy of process and the final cost of the extract obtained.

The extracts obtained by the process of the invention are characterised by a high caffeoylquinic acid content, and exert a potent hypoglycaemic activity on the postprandial and baseline blood glucose levels. Said effect is also maintained if the product is added in suitable amounts to foods rich in carbohydrates, which is the major application of this novel extract in the dietary field.

Heat treatment used in desolvation together with acid treatment at the extraction step induces structural modifications that lead to improved biological activity of the extract in terms of its antioxidant and metabolic effect. The treatment cleaves bonds with protein structures, wherein caffeoylquinic acids, changing to the quinone form, bind to the SH groups of proteins with the Michael reaction or reactions with amino groups which often accompany the fate of polyphenols in plants.

The Helianthus annuus extract obtained by the process according to the invention preferably has a caffeoylquinic acid content ranging from 40 to 80%, preferably from 50 to 60%.

The extract of the invention can be advantageously formulated for human treatment as oils enriched with diglycerides, in the presence or absence of phospholipids as surfactant carrier, or incorporated in foods such as bread, all types of biscuits, and foods in general which do not undergo aqueous washing at high temperature, because the active ingredients are freely water-soluble. In view of the latter aspect, the caffeoylquinic acids could be made insoluble in water by forming complexes with vegetable or animal proteins which, when denatured by heat, incorporate them in a stable manner. The active products are released in the intestine by enzymatic hydrolysis of the protein, where they can interact with other substrates and modify the absorption of glucose, inhibiting the enzyme 6-phosphate synthetase.

It has been observed that the addition of the extract to a food rich in starchy carbohydrates significantly reduces the postprandial blood glucose level.

According to the present invention, the amount of extract to be administered as such in nutraceutical formulations generally ranges between 50 and 500 mg, preferably 250 mg, at each meal at which starchy carbohydrates are eaten.

The results of the clinical trial are set out below.

Postprandial Blood Glucose Level

The subjects were given, under controlled clinical trial conditions, a mixed Mediterranean meal containing 60% carbohydrates, 25% lipids and 15% proteins, together with 250 mg of the extract according to the invention. An 18% reduction in the postprandial blood glucose level was observed (p≦0.05) (12 volunteers vs. placebo).

Baseline Blood Glucose Level

The trial subjects, who were healthy volunteers, were treated for one month with three capsules containing 250 mg of extract (at breakfast, lunch and dinner), which they took with a standard Mediterranean diet (see above), which was equal for the different subjects in the placebo-controlled crossover study. At the end of the month's treatment, a 15% reduction in the baseline blood glucose level was observed (subjects with a borderline baseline blood glucose level of 110±5).

Enhancement of postprandial and fasting hypoglycaemic activity makes these extracts a useful modulator of the body weight and metabolic syndrome in all cases wherein an incorrect diet or dysmetabolism associated with age has created health problems.

A reduction in the blood triglyceride level was also observed In the treated patients. In separate clinical tests on subjects suffering from liver disease with elevated transaminase values, the treatment reduced said parameters to normal, with an evident reduction in liver steatosis.

As already mentioned, under suitable conditions the extracts according to the invention can react rapidly with macromolecules, especially glycoproteins, which involves two advantages. Firstly, the extracts complexed with macromolecules are protected against bacterial attack and oxidation and are released, after their enzymatic or bacterial demolition, in sites where they can perform their hypoglycaemic and antioxidant activity. Secondly, the extracts complexed with macromolecules can also be used in aqueous environments. In this way, they can be added to foods like pasta (which must be cooked in water) without any appreciable loss of active ingredients.

The extracts of the invention can also be added to bread, pizza, rusks, biscuits, drinks and foods in general, including those based on proteins.

According to another preferred aspect, the extracts of the invention are formulated as conventional or gastroprotected capsules or tablets so as to promote topical local activity, leaving the digestive function unchanged at stomach level. According to a preferred aspect, the formulations containing the extracts according to the invention will be supplemented with oils rich in diglycerides.

According to a further aspect, the compositions according to the invention can also contain other substances with a useful or complementary activity.

The compositions according to the invention are formulated by conventional methods, such as those described in “Remington's Pharmaceutical Handbook”, Mack Publishing Co., N.Y., USA. In particular, the compositions according to the invention are formulated by conventional formulation techniques used for vegetable ingredients, which require particular care to be taken to avoid interactions with the excipients and the capsule matrices. Examples of oral formulations are tablets, dragées, soft and hard gelatin capsules, and cellulose capsules.

The examples set out below further illustrate the invention.

### EXAMPLE 1

### Preparation of Helianthus Annuus Extract by Nanofiltration

10 Kg of deoiled Helianthus annuus seeds is pelletted and extracted with an 85% v/v mixture of ethanol/water containing a amount of H₂SO₄ sufficient to maintain the pH at 2.5, until the caffeoylquinic acid content is exhausted. Extraction is performed at a temperature of 40° C. The water-alcohol solution is concentrated to 10 L “until complete elimination of ethanol”, and products insoluble in water are then filtered. The aqueous solution is alkalinised to pH 5 and then subjected to ultrafiltration using a 10 KDa flat organic membrane. The perfectly clear solution containing all the caffeoylquinic acids, flavonoids and other polyphenols in small amounts then undergoes nanofiltration through a ceramic membrane with a 400 Da cut-off. The caffeoylquinic acids are concentrated in the retentate, while the permeate, which contains salts, sugars and undesirable low-molecular-weight products, is discarded. The retentate is concentrated to a dry residue of 10% and atomised. 1.2 kg of a pale beige extract is obtained, which has a caffeoylquinic acid content of 56%, measured by HPLC, and a chlorogenic acid content of 32%. This extract is used to prepare capsules or tablets, or can be added to various foods in suitable doses.

### EXAMPLE 2

### Preparation of Helianthus Annuus Extract by Chromatography

50 Kg of deoiled Helianthus annuus seeds is pelletted and extracted with an 85% v/v mixture of ethanol/water containing a amount of H₂SO₄ sufficient to maintain the pH at 2.5, until the caffeoylquinic acid content is exhausted. Extraction is performed at a temperature of 40° C. The residual biomass is discarded, and the water-alcohol solution is concentrated until the ethanol is eliminated. The aqueous solution is concentrated to 10 L, and the water-insoluble products are filtered. The aqueous solution is alkalinised to pH 5 and subjected to ultrafiltration through an organic membrane with a 10 KDa cut-off. The clear aqueous concentrate is absorbed on 50 L of a polystyrene absorbing resin from which the active extract is subsequently recovered by elution of the resin with 90% ethanol/water.

After concentration until dry, about 4 kg of extract containing 56% caffeoylquinic acids, expressed as chlorogenic acids, is obtained.

### EXAMPLE 3

### Cellulose Capsules

Type 0 Cellulose Capsules are Filled with the Following Ingredients:

Unit Composition:

| | | |
|---|---|---|
| Helianthus annuus extract | 250 | mg |
| Soya lecithin | 10 | mg |
| Sunflower oil | q.s. for 700 | mg |

### EXAMPLE 4

### Tablets

Unit Composition:

| | |
|---|---|
| Helianthus annuus extract | 200 mg |
| Microcrystalline cellulose | 300 mg |
| Lactose | 190 mg |
| Silicon dioxide | 5 mg |
| Magnesium stearate | 5 mg |

### EXAMPLE 5

### Food Preparation (Pizza)

About 200 g of flour is mixed with 10 g of brewer's yeast, salt, oil and 50 ml of water. The ingredients are kneaded, 500 mg of Helianthus annuus extract is added, and the dough is left to stand for 2 h. The dough is then rolled out, cheese and other desired ingredients added, and the pizza is cooked in a hot oven at 200° C. until ready. The glycaemic index of this pizza was compared with that of a pizza prepared with the same ingredients but without the addition of Helianthus annuus extract, and the glycaemic index was 15% lower.

1. A process for the preparation of extracts of Helianthus annuus, which comprises:
a) extracting with aqueous mixtures of aliphatic alcohols Helianthus annuus seeds obtained by extraction with hexane followed by solvent elimination at temperatures above 100° C.;
b) concentrating the water-alcohol solution from step a) under vacuum to complete elimination of the alcohol solvent, and filtering any residual insolubles and fatty phases;
c) adjusting the pH of the aqueous solution from step b) to pH 5;
d) subjecting the aqueous solution from step c) to ultrafiltration on 10 kDa organic membranes;
e) subjecting the solution from step d) to chromatography or nanofiltration;
f) concentrating the retentate from step e) under vacuum or by atomisation.

2. The process of claim 1, wherein in step a) the extraction is carried out with ethanol/water mixtures, in the presence of organic or inorganic acids capable of maintaining a pH below 2.

3. The process of claim 2, wherein in step a) the extraction is carried out with 80% v/v ethanol/water mixtures in the presence of dilute sulphuric acid.

4. The process of claim 1, wherein in step c) the pH of the aqueous solution is adjusted to values around 5 using calcium carbonate.

5. The process of claim 1, wherein in step e) the solution is subjected to chromatography on absorption resin using a polystyrene resin and/or ion exchange and absorption resin.

6. The process of claim 1, wherein in step e) the solution is subjected to nanofiltration using a ceramic membrane with cut-off from 400 to 600 Da.

7. Extracts of Helianthus annuus obtained with the process of claim 1.

8. The extracts of Helianthus annuus of claim 7, having a caffeoylquinic acid content ranging from 40 to 80%, preferably from 50 to 60%.

9. The extracts of Helianthus annuus of claim 7, complexed with vegetable or animal proteins.

10. Formulations comprising the extracts of Helianthus annuus of claim 7.

11. The formulations of claim 10 containing 50 to 500 mg of extracts of Helianthus annuus.

12. The formulations of claim 9, also containing oils enriched with diglycerides and optionally surfactants.

13. The formulations of claim 10 in the form of conventional or gastro-protected capsules or tablets.

14. Foods based on carbohydrates containing the extracts of claim 7.
